(19) United States
(12) Patent Application Publication
(10) Pub. No.: US 2021/0212550 A1
(43) Pub. Date: Jul. 15, 2021

(54) ENDOSCOPE APPARATUS, PROCESSING STATE SWITCHING METHOD, NETWORK APPARATUS AND MEDICAL SYSTEM

(71) Applicant: OLYMPUS CORPORATION, Tokyo (JP)

(72) Inventor: Misa HIROSE, Hachioji-shi (JP)

(73) Assignee: OLYMPUS CORPORATION, Tokyo (JP)

(21) Appl. No.: 17/213,786

(22) Filed: Mar. 26, 2021

Related U.S. Application Data
(63) Continuation of application No. PCT/JP2019/037833, filed on Sep. 26, 2019.

(30) Foreign Application Priority Data
Sep. 28, 2018 (JP) 2018-183187

(51) Int. Cl. A61B 1/00

(52) U.S. Cl. CPC A61B 1/00011; A61B 2560/0487; A61B 1/00039; A61B 1/00004

(57) ABSTRACT

An endoscope apparatus includes a connection interface to which an endoscope is connected, a communication interface that transmits/receives signals to/from an external apparatus, a computation device that determines whether or not the endoscope is connected to the connection interface, switches a processing state of the endoscope apparatus from an inspection state to a non-inspection state or from a non-inspection state to an inspection state, and performs control to switch the processing state from the inspection state to the non-inspection state upon acquiring an instruction signal for instructing switching of the processing state from the inspection state to the non-inspection state from the external apparatus via the communication interface, when it is determined that the endoscope is not connected to the connection interface.

### CROSS-REFERENCE TO RELATED APPLICATIONS

This application is based upon and claims the benefit of priority from prior Japanese Patent Application No. 2018-183187, filed Sep. 28, 2018, the entire contents of which are incorporated herein by this reference.

This application is a continuation application of International Application PCT/JP2019/037833 filed on Sep. 26, 2019, the entire contents of which are incorporated herein by reference.

### TECHNICAL FIELD

The present embodiment relates to an endoscope apparatus, a processing state switching method executed in the endoscope apparatus, a network apparatus and a medical system including the endoscope apparatus and the network apparatus.

### BACKGROUND

Conventionally, endoscope systems used for endoscope inspection switch a processing state of the endoscope systems between an inspection state and a non-inspection state depending on input of an inspection start instruction or an inspection end instruction. This makes it possible to associate patient information inputted to the endoscope systems prior to an inspection with inspection images taken during the inspection, and separate and manage the patient information and the inspection images for each inspection. Note that since the endoscope apparatus included in the endoscope system can control the entire operation of the endoscope system, the processing state of the endoscope system can be said to be the processing state of the endoscope apparatus.

FIG. 8 is a diagram illustrating a switching example of the processing state of an endoscope system.

Note that FIG. 8 illustrates a case where the processing state of the endoscope system is an inspection state as “under inspection” and a case where the processing state of the endoscope system is a non-inspection state as “not under inspection.”

In the endoscope system shown in FIG. 8, while the processing state is a non-inspection state, if a user such as a medical doctor or nurse registers patient information of the next inspection (e.g., patient information a of patient A) and inputs an inspection start instruction, the processing state is switched from the non-inspection state to the inspection state.

Here, a registration of the patient information is performed through, for example, operation of a keyboard connected to the endoscope apparatus. Alternatively, a registration of the patient information may also be performed by inputting the patient information using a barcode reader, a magnetic card reader or a medical tablet or the like. An inspection start instruction is executed, for example, by pressing an inspection start key of the endoscope apparatus or by pressing a still image recording button or a capture button in the non-inspection state at the first time. The still image recording button or the capture button is provided at an operation section of the endoscope (also referred to as a “scope”) connected to the endoscope apparatus. Alternatively, the inspection start instruction may also be executed by pressing the recording button in the non-inspection state at the first time according to the user's setting. The recording button is also provided at the operation section of the endoscope. Alternatively, the inspection start instruction may also be executed by an operation on the front panel of the endoscope apparatus or the medical tablet.

After the processing state of the endoscope system is switched to the inspection state, inspection images are taken, for example, by the user pressing the still image recording button.

When the user inputs an inspection end instruction, the processing state of the endoscope system is switched from the inspection state to the non-inspection state, the patient information inputted before the inspection (e.g., patient information a of patient A) and inspection images taken under inspection are associated with each other and stored.

Here, the inspection end instruction is executed, for example, by pressing the inspection end key of the endoscope apparatus, registering the patient information of the next inspection (e.g., patient information b of patient B), that is, updating the patient information. Alternatively, the inspection end instruction can also be executed by turning power from OFF to ON through operation of a power button of the endoscope apparatus according to the user's setting or removing the endoscope from the endoscope apparatus, that is, disconnecting the endoscope from the endoscope apparatus.

In this way, in the endoscope system shown in FIG. 8, registration of patient information, an inspection start instruction or an inspection end instruction is executed through operation of the keyboard connected to the endoscope apparatus, key operation of the endoscope apparatus, button operation of the operation section of the endoscope connected to the endoscope apparatus or the like. That is, registration of patient information, inspection start instruction or inspection end instruction is executed in an inspection room in which the endoscope system is provided.

Note that various proposals regarding apparatuses and systems used in the medical field have been made.

For example, WO2012/043095 proposes a medical system constructed of a medical imaging apparatus and a server that records medical images transmitted from the medical imaging apparatus, the medical system including first inspection state storing means and second inspection state storing means for storing an inspection state of “under inspection” or “inspection end” of the medical imaging apparatus in the medical imaging apparatus and the server respectively, first control means provided with a function to notify the server of the inspection state of the medical imaging apparatus and a function to shift the medical imaging apparatus to the inspection end according to an inspection end execution instruction from the server and second control means for generating an inspection end execution instruction for changing the inspection state of the medical imaging apparatus from the server based on information on the inspection state received from the medical imaging apparatus and a state of the server.

For example, Japanese Patent Application Laid-Open No. 2014-188082 proposes a medical information management apparatus connected to a medical instrument system via a communication channel, the medical information management apparatus including an order holding section that holds order of medical actions that should be executed using a medical instrument system, an order management section that manages order statuses, an order notification section that notifies the medical instrument system of the order and a receiving section that receives an end notification of a medical action defined in a certain order from the medical instrument system, in which when the receiving section receives an end notification of a medical action, the order management section changes the status of the order held in the order holding section that defines the medical action to “already executed,” the order management section extracts an order, a status of which is not executed yet, from among the orders held in the order holding section in response to a request to acquire the order from the medical instrument system, extracts an order within a set time after the status changes to “already executed,” and causes the order notification section to notify the medical instrument system of the extracted order.

### SUMMARY

One aspect of the present embodiment is an endoscope apparatus including a connection interface to which an endoscope is connected, a communication interface that transmits/receives signals to/from an external apparatus, and a computation device that determines whether or not the endoscope is connected to the connection interface, switches a processing state of the endoscope apparatus from an inspection state to a non-inspection state or from a non-inspection state to an inspection state, and performs control to switch the processing state from the inspection state to the non-inspection state upon acquiring an instruction signal for instructing switching of the processing state from the inspection state to the non-inspection state from the external apparatus via the communication interface, when it is determined that the endoscope is not connected to the connection interface.

Another aspect of the present embodiment is a processing state switching method executed by an endoscope apparatus, the method including determining whether or not an endoscope is connected to the endoscope apparatus upon acquiring an instruction signal representing an instruction to switch the processing state of the endoscope apparatus from an inspection state to a non-inspection state from an external apparatus and switching the processing state of the endoscope apparatus from an inspection state to a non-inspection state when it is determined that the endoscope is not connected to the endoscope apparatus.

A further aspect of the present embodiment is a medical system including an endoscope apparatus and a network apparatus, the endoscope apparatus including a connection interface to which an endoscope is connected, a first computation device that determines whether or not the endoscope is connected to the connection interface, switches a processing state of the endoscope apparatus from an inspection state to a non-inspection state or from a non-inspection state to an inspection state and controls the switching, and a first communication interface that transmits/receives signals to/from the network apparatus, in which the network apparatus includes a second communication interface that transmits/receives signals to/from the endoscope apparatus, a second computation device that determines whether or not the endoscope is disconnected from the endoscope apparatus, an output device that announces to the user that the endoscope is disconnected from the endoscope apparatus when the second computation device determines that the endoscope is disconnected from the endoscope apparatus, and an input device that inputs an instruction, the first communication interface transmits a notification signal for notifying a determination result of the first computation device to the network apparatus, the second computation device makes the determination based on the notification signal acquired from the endoscope apparatus via the second communication interface, the second communication interface transmits an instruction signal for instructing switching of the processing state from the inspection state to the non-inspection state to the endoscope apparatus when an inspection end instruction is inputted via the input device, and the first computation device performs control to switch the processing state from the inspection state to the non-inspection state when the instruction signal is acquired from the network apparatus via the first communication interface.

A still further aspect of the present embodiment is an endoscope apparatus including a connection interface to which an endoscope is connected, a computation device that determines whether or not the endoscope is connected to the connection interface, switches a processing state of the endoscope apparatus from an inspection state to a non-inspection state or from a non-inspection state to an inspection state, and controls the switching and a communication interface that transmits/receives signals to/from an external apparatus, in which the communication interface transmits a notification signal for notifying a determination result of the computation device to the external apparatus and the computation device performs control to switch the processing state from the inspection state to the non-inspection state when an instruction signal for instructing switching of the processing state from the inspection state to the non-inspection state is acquired from the external apparatus via the communication interface.

A still further aspect of the present embodiment is a network apparatus including a communication interface that transmits/receives signals to/from an endoscope apparatus, a computation device that determines whether or not an endoscope is disconnected from the endoscope apparatus, an output device that announces to a user that the endoscope is disconnected from the endoscope apparatus when the computation device determines that the endoscope is disconnected from the endoscope apparatus, and an input device that inputs an instruction, in which the computation device makes the determination based on a notification signal for notifying the connection interface of the endoscope apparatus of a determination result as to whether or not the endoscope is connected acquired from the endoscope apparatus via the communication interface and the communication interface transmits an instruction signal representing an instruction for switching a processing state of the endoscope apparatus from an inspection state to a non-inspection state to the endoscope apparatus when an inspection end instruction is inputted via the input device.

### BRIEF DESCRIPTION OF DRAWINGS

FIG. 1 is a diagram illustrating a configuration example of a medical system according to a first embodiment.

FIG. 2 is a diagram illustrating an example of a hardware configuration relating to a processing state switching function provided for a video processor.

FIG. 3 is a diagram illustrating an example of a hardware configuration of a server.

FIG. 4 is a diagram schematically illustrating an example of processing relating to a processing state switching function executed by a video processor and processing executed by the server.

FIG. 5 is a diagram more schematically illustrating processing relating to the processing state switching function.

FIG. 6 is a diagram schematically illustrating an example of processing executed by the video processor and the server in a medical system according to a modification.

FIG. 7 is a diagram schematically illustrating an example of processing executed by a medical system according to a second embodiment.

FIG. 8 is a diagram illustrating a switching example of a processing state of an endoscope system.

FIG. 9 is a diagram illustrating an example of a case where an inspection end instruction is executed remotely.

FIG. 10 is a diagram illustrating an example of a case where patient information is registered remotely.

### DESCRIPTION OF EMBODIMENTS

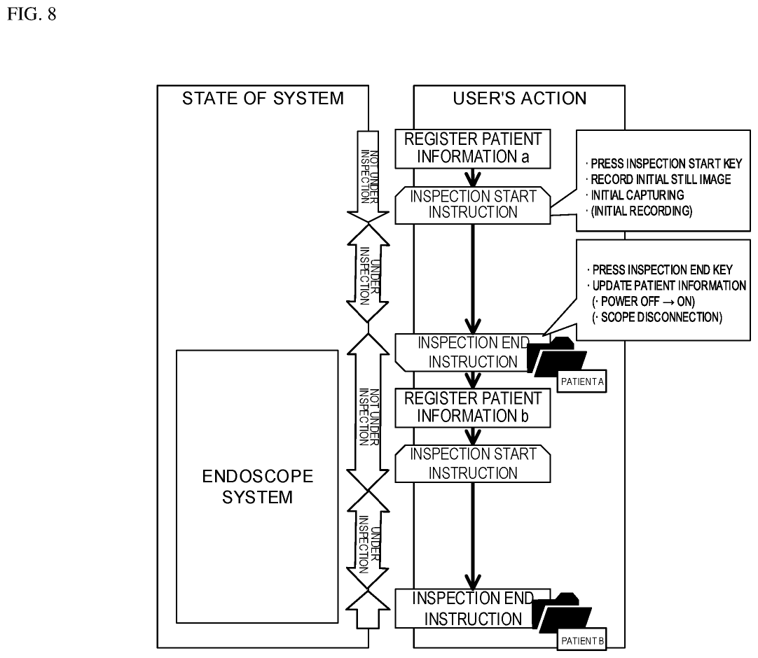

In an endoscope system shown in FIG. 8, registering patient information, instructing an inspection start and instructing an inspection end remotely via a network are prohibited in principle. Describing an inspection end instruction as an example, this is for the following reasons.

Assuming that a remote inspection end instruction is allowed, a remote user basically cannot grasp a situation in an inspection room, and so an inspection end instruction may be executed despite the inspection being still in progress in the inspection room, and the processing state of the endoscope system may be switched from an inspection state to a non-inspection state in an unintended situation.

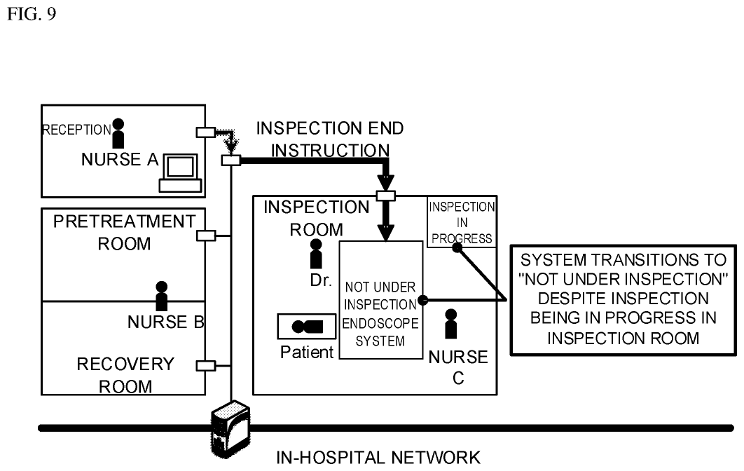

FIG. 9 is a diagram illustrating an example of case where an inspection end instruction is executed remotely.

In the example shown in FIG. 9, a user (e.g., nurse A) in the reception executes an inspection end instruction from a terminal in the reception on the endoscope system in the inspection room via an in-hospital network despite the inspection being still in progress in the inspection room, and as a result, the processing state of the endoscope system is switched from the inspection state to the non-inspection state in an unintended situation.

On the other hand, some medical facilities may allow only a remote registration of patient information via a network. However, this may interfere with smooth progress of inspection as follows.

For example, if the user in the inspection room forgets to execute an inspection end instruction, the processing state of the endoscope system may still be considered as the inspection state despite the inspection having actually ended. Since a registration of patient information is allowed when the processing state of the endoscope system is a non-inspection state, the registration of patient information is not allowed if the processing state of the endoscope system is still an inspection state. Therefore, in this case, the remote user in charge of the registration of patient information needs to go to the inspection room and execute an inspection end instruction or the like.

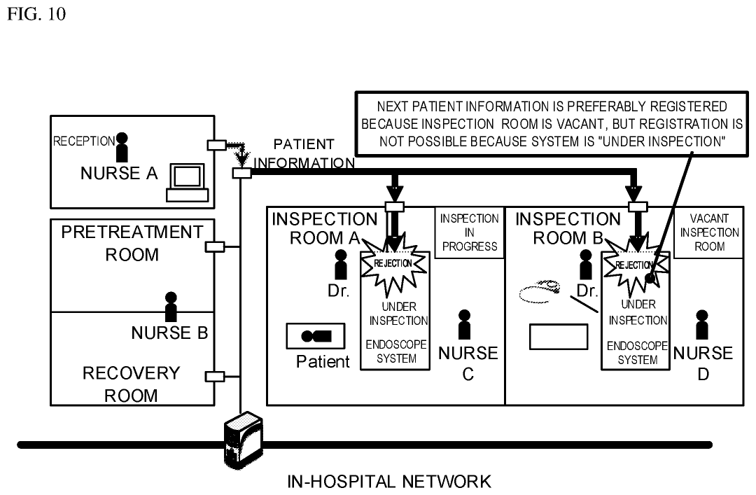

FIG. 10 is a diagram illustrating an example of a case where a registration of patient information is remotely performed.

In the example shown in FIG. 10, an inspection is in progress in an inspection room A and the processing state of an endoscope system provided in the inspection room A is an inspection state. On the other hand, as for an inspection room B, a user (e.g., Dr. or Nurse D) in the inspection room B forgets to execute an inspection end instruction despite the inspection room B being vacant with an inspection already ended, and so the processing state of the endoscope system provided in the inspection room B is still an inspection state.

In this case, when the user (e.g., Nurse A) in the reception performs a registration of patient information with the endoscope system provided in the inspection room A from a terminal in the reception via the in-hospital network, since the processing state of the endoscope system is an inspection state, the registration of patient information is rejected. In this case, the rejection of the registration of patient information is appropriate because an inspection is in progress in the inspection room A.

On the other hand, when the user (e.g., Nurse A) in the reception performs a registration of patient information with the endoscope system provided in the inspection room B from the terminal in the reception via the in-hospital network, the registration of patient information is rejected because the processing state of the endoscope system is still an inspection state. In this case, since the inspection room B is vacant, the registration of patient information should originally be allowed, but since the processing state of the endoscope system is still an inspection state, the registration of patient information is not allowed. Thus, in this case, the user in the reception has to go to the inspection room B, execute an inspection end instruction or the like and switch the processing state of the endoscope system to a non-inspection state.

Thus, in embodiments disclosed hereinafter, an endoscope apparatus, a processing state switching method, a network apparatus and a medical system will be provided, which can appropriately switch a processing state of the endoscope apparatus from an inspection state to a non-inspection state remotely according to an inspection end instruction.

Hereinafter, the embodiments will be described with reference to the accompanying drawings.

### First Embodiment

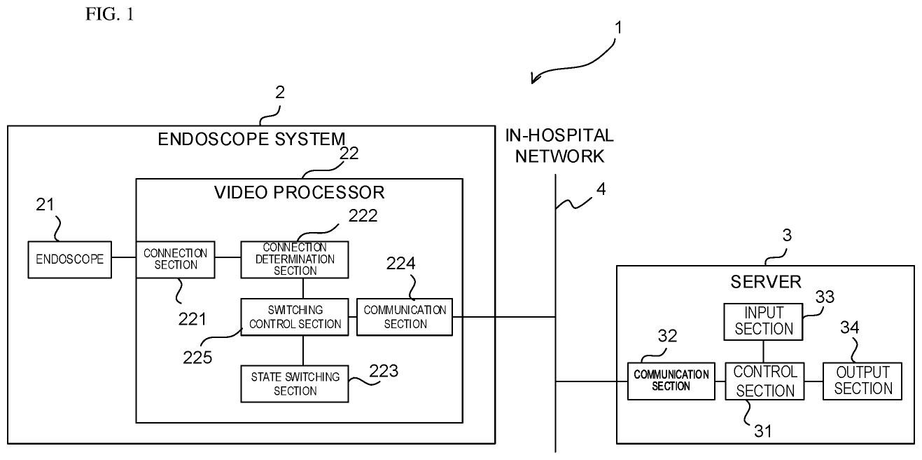

FIG. 1 is a diagram illustrating a configuration example of a medical system according to a first embodiment.

In FIG. 1, a medical system 1 is a system provided in a hospital and includes an endoscope system 2 installed in an inspection room in the hospital, a server 3 installed outside the inspection room in the hospital and an in-hospital network 4.

The endoscope system 2 includes an endoscope 21 and a video processor 22, and the video processor 22 is configured to allow connection of the endoscope 21. Although not shown, the endoscope system 2 further includes a monitor or a peripheral device and the video processor 22 is configured to allow connection of a monitor and a peripheral device as well. An example of the peripheral device is an image recording apparatus.

Part of the endoscope 21 is inserted into a subject to pick up images of the subject and output an image pickup signal. The endoscope 21 is provided with an image pickup device to pick up images of the subject. An example of the image pickup device is a CCD (charge coupled device).

The video processor 22 is an example of the endoscope apparatus and controls overall operation of the endoscope system 2. The video processor 22 applies, for example, predetermined signal processing to the image pickup signal outputted from the endoscope 21, thereby generates image data and processes the generated image data such as displaying the generated image data on a monitor connected to the video processor 22 or recording the generated image data in an image recording apparatus connected to the video processor 22.

The video processor 22 can switch the own processing state between an inspection state and a non-inspection state. Here, the inspection state shows a state in which the video processor 22 performs processing such as generating image data and recording the generated image data and patient information relating to a patient under inspection in association with each other. The video processor 22 has a function capable of switching the own processing state from an inspection state to a non-inspection state (hereinafter referred to as a “processing state switching function”) according to an inspection end instruction signal from an external apparatus such as the server 3. The inspection end instruction signal here is also a signal for instructing switching of the processing state of the video processor 22 from the inspection state to the non-inspection state.

The video processor 22 is provided with a connection section 221, a connection determination section 222, a state switching section 223, a communication section 224 and a switching control section 225 as components related to the processing state switching function.

The connection section 221 is a connection interface to which the endoscope 21 is connected. The connection section 221 (connection interface) is configured so that the connector of the endoscope 21 (not shown in the figure) can be attached and detached.

The connection section 221 is electrically connected to the endoscope 21 and performs various communications.

The endoscope 21 has, for example, an image signal line, which transmits the image signal of the image captured by the image sensor to the video processor 22. The video processor 22 receives the image signal through the connection interface.

The communication section 224 (communication interface) of the video processor 22 and the communication section 32 of the server 3 are connected via the network 4. Network 4 is a communication channel using, for example, the Internet or a wireless LAN, and can include a dedicated line (dedicated cable) for direct connection, a LAN using Ethernet (registered trademark), etc., as well as a communication network such as a telephone communication network, cable network, or wireless LAN. The communication method can be wired or wireless.

The connection determination section 222 determines whether or not the endoscope 21 is connected to the connection section 221. This determination is performed, for example, by detecting whether or not the endoscope 21 is connected to the connection section 221.

The state switching section 223 can switch the processing state of the video processor 22 between the inspection state and the non-inspection state.

The communication section 224 is a communication interface that transmits/receives signals to/from an external apparatus such as the server 3 via the in-hospital network 4. For example, the communication section 224 receives an inspection end instruction signal from the server 3 or transmits an inspection end notification signal or an inspection end disable notification signal to the server 3. The inspection end notification signal here is also a notification signal for notifying that the endoscope system 2 is not under inspection and the inspection end disable notification signal is also a notification signal for notifying that the endoscope system 2 is under inspection. Regarding communication of signals with an external apparatus such as the server 3 via the in-hospital network 4, signals may be transmitted/received in an encrypted state.

When the switching control section 225 acquires an inspection end instruction signal from the server 3 via the communication section 224, if the connection determination section 222 determines that the endoscope 21 is not connected to the connection section 221, the switching control section 225 performs control over the state switching section 223 so as to switch the processing state from the inspection state to the non-inspection state. Note that in this case, an inspection end notification signal is transmitted from the communication section 224 to the server 3. On the other hand, when the switching control section 225 acquires an inspection end instruction signal, if the connection determination section 222 determines that the endoscope 21 is connected to the connection section 221, the switching control section 225 does not perform control over the state switching section 223 so as to switch the processing state from the inspection state to the non-inspection state. Note that in this case, an inspection end disable notification signal is transmitted from the communication section 224 to the server 3.

The server 3 is an example of the external apparatus with respect to the video processor 22, and is also an example of the network apparatus. The server 3 is provided with a control section 31, a communication section 32, an input section 33 and an output section 34.

The control section 31 controls overall operation of the server 3.

The communication section 32 is a communication interface that transmits/receives signals to/from an external apparatus such as the video processor 22. For example, upon receiving an inspection end instruction via the input section 33, the communication section 32 transmits an inspection end instruction signal to the video processor 22. For example, the communication section 32 receives an inspection end notification signal or an inspection end disable notification signal from the video processor 22.

The input section 33 is, for example, a keyboard, a mouse or a touch panel, and is an input device that performs various types of input according to the user's operation. For example, the input section 33 inputs an inspection end instruction according to the user's operation.

The output section 34 is, for example, a display apparatus or a printing apparatus, and is an output device that outputs various information. For example, the output section 34 performs display to announce an inspection end to the user when the communication section 32 receives an inspection end notification signal from the video processor 22 or performs display to announce inspection end disable to the user when the communication section 32 receives an inspection end disable notification signal from the video processor 22.

The in-hospital network 4 is a wired network, a wireless network or a network where a wired channel and a wireless channel are mixed, and is, for example, a LAN (local area network).

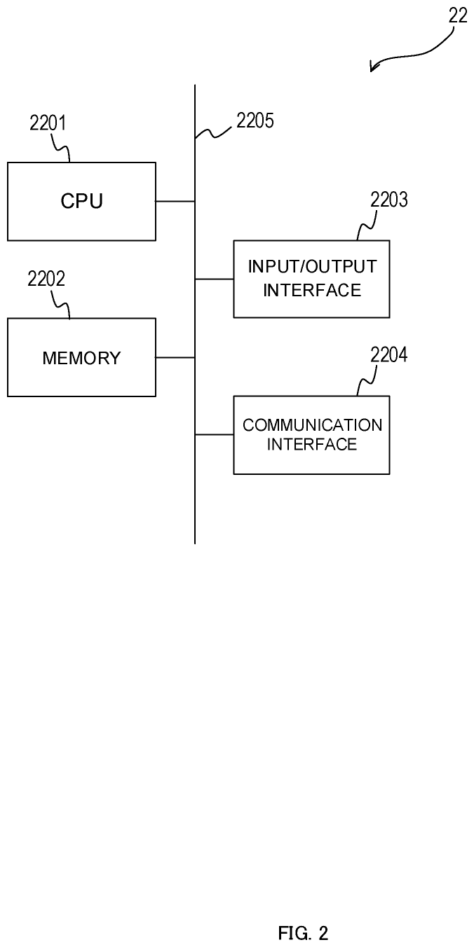

FIG. 2 is a diagram illustrating an example of a hardware configuration relating to a processing state switching function provided for the video processor.

As shown in FIG. 2, the video processor 22 is provided with a CPU 2201, a memory 2202, an input/output interface 2203 and a communication interface 2204 as a hardware configuration relating to a processing state switching function, and these are mutually connected via a bus 2205.

The CPU 2201 is a computation device that executes a program to perform processing relating to the processing state switching function. The memory 2202 is a RAM (random access memory) and a ROM (read only memory), and the RAM is used as a work area for the CPU 2201 and the ROM stores a program and information necessary to execute the program in a non-volatile manner.

The input/output interface 2203 is connected to the endoscope 21 or the like and configured to input/output signals from/to the endoscope 21 or the like. The communication interface 2204 is connected to the in-hospital network 4 and configured to communicate with an external apparatus such as the server 3.

Note that the processing state switching function provided by the video processor 22 is not limited to the hardware configuration shown in FIG. 2, but may also be implemented by a circuit such as an FPGA (field-programmable gate array) or an ASIC (application specific integrated circuit).

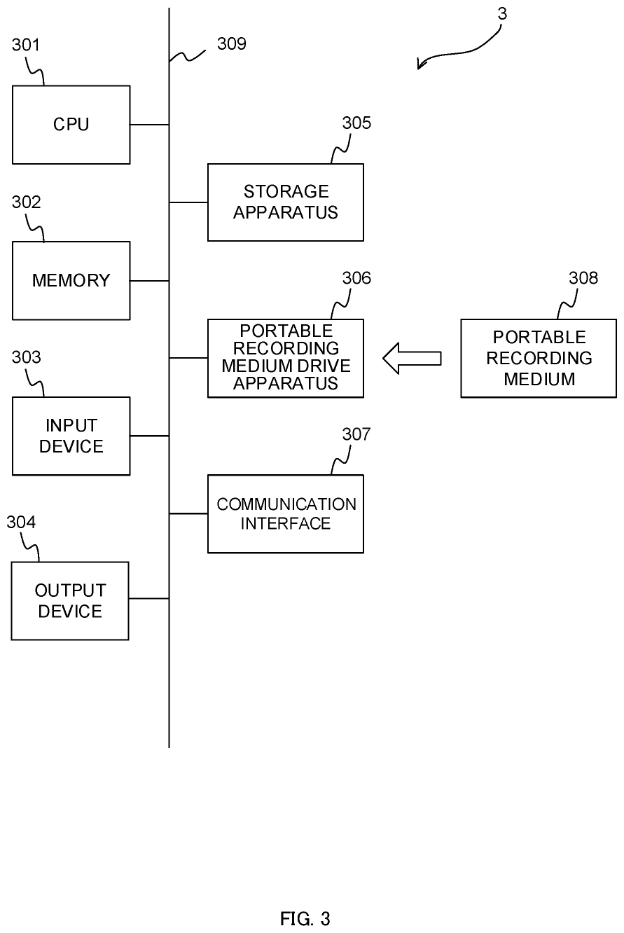

FIG. 3 is an example of a hardware configuration of the server.

As shown in FIG. 3, the server 3 is provided with a CPU 301, a memory 302, an input device 303, an output device 304, a storage apparatus 305, a portable recording medium drive apparatus 306 in which a portable recording medium 308 is housed and a communication interface 307, all of which are mutually connected via a bus 309.

The CPU 301 is a computation device that executes a program for processing executed by the server 3. The memory 302 is a RAM and a ROM, the RAM is used as a work area or the like for the CPU 301 and the ROM stores a program and information necessary to execute the program in a non-volatile manner.

The input device 303 is a touch panel, a keyboard or the like, and used to input an instruction or information from the user. The output device 304 is a display apparatus, a printing apparatus or the like, and used to output various types of information.

The storage apparatus 305 is a storage that stores a program and information necessary to execute the program and information acquired by executing the program or the like in a non-volatile manner. The storage apparatus 305 is, for example, a hard disk apparatus. The portable recording medium drive apparatus 306 drives the portable recording medium 308 and accesses recording contents thereof. The portable recording medium 308 is a memory device, a flexible disk, an optical disk, a magneto-optical disk or the like. Examples of the portable recording medium 308 include a CD-ROM (compact disk read only memory), a DVD (digital versatile disk), a USB (universal serial bus) memory or the like. Just like the storage apparatus 305, the portable recording medium 308 stores a program and information necessary to execute the program and information acquired by executing the program or the like in a non-volatile manner.

The communication interface 307 is connected to the in-hospital network 4 and communicates with an external apparatus such as the video processor 22.

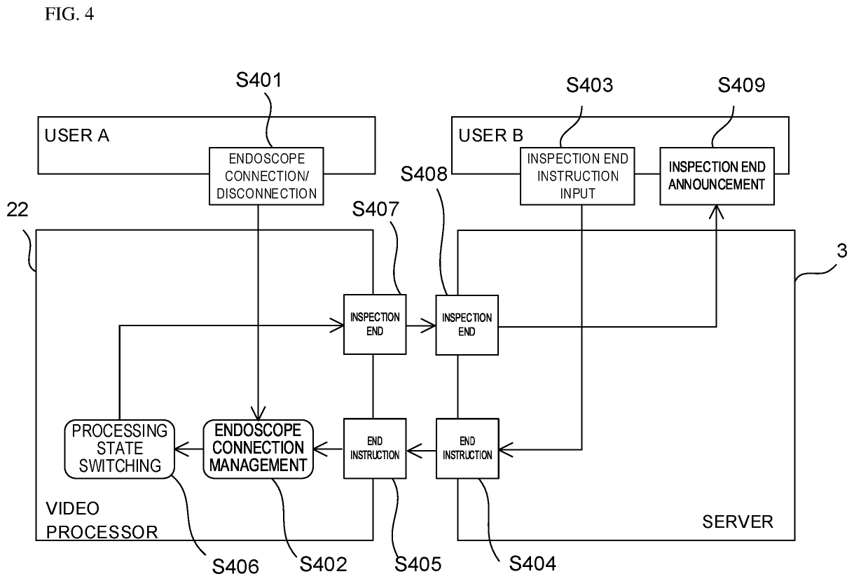

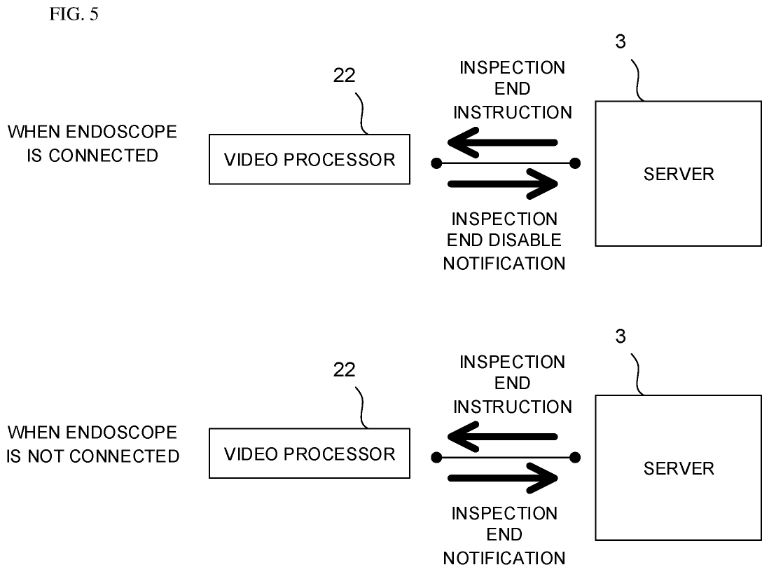

Next, processing relating to the processing state switching function executed by the video processor 22 will be described using FIG. 4 and FIG. 5.

FIG. 4 is a diagram schematically illustrating an example of processing relating to a processing state switching function executed by the video processor and processing executed by the server. FIG. 5 is a diagram more schematically illustrating processing relating to the processing state switching function at this time.

In the processing shown in FIG. 4, when a user A (e.g., medical doctor or nurse) in the inspection room connects or disconnects the endoscope 21 to/from the video processor 22 (S401), the connection determination section 222 determines the connection or disconnection, and the video processor 22 stores and manages the determination result as a current connection state of the endoscope 21 (S402).

When a user B (e.g., nurse) outside the inspection room inputs an inspection end instruction to the server 3 (S403), the server 3 transmits an inspection end instruction signal to the video processor 22 (S404). Note that the processing state of the video processor 22 at this time is an inspection state.

When the video processor 22 receives an inspection end instruction signal from the server 3 (S405), when it is determined based on the current connection state of the endoscope 21 under management that the endoscope 21 is not connected (S402), the video processor 22 switches the own processing state from the inspection state to the non-inspection state (S406), and transmits an inspection end notification signal to the server 3 (S407). Note that in the inspection room, when the inspection ends, since the endoscope 21 is disconnected from the video processor 21, if the endoscope 21 is not connected yet, the inspection can be regarded as having ended.

When the server 3 receives the inspection end notification signal from the video processor 22 (S408), the server 3 announces an inspection end, that is, that the endoscope system 2 is not under inspection to the user B (S409). The announcement is performed, for example, by display. The user B who has confirmed that the endoscope system 2 is not under inspection by the announcement, then performs a registration of patient information for the next inspection. The registration of patient information is also performed on the video processor 22 via the server 3 just like the inspection end instruction.

On the other hand, although not shown, when the video processor 22 receives an inspection end instruction signal from the server 3 (S405), when it is determined from the current connection state of the endoscope 21 under management that the endoscope 21 is connected (S402), the video processor 22 transmits an inspection end disable notification signal to the server 3 without switching the own processing state. Upon receiving the inspection end disable notification signal from the video processor 22, the server 3 announces inspection end disable, that is, that the endoscope system 2 is under inspection to the user B. The user B who has confirmed that the endoscope system 2 is under inspection by the announcement, does not perform a registration of patient information for the next inspection at least until confirming that the endoscope system 2 is not under inspection.

Note that upon receiving an inspection end instruction signal from the server 3 (S405), the connection determination section 222 of the video processor 22 may determine at that timing that the endoscope 21 is connected to the connection section 221 and switch the processing state from the inspection state to the non-inspection state according to the determination result. That is, the video processor 22 may switch the processing state when it is determined that the endoscope 21 is not connected and may not switch the processing state when it is determined that the endoscope 21 is connected.

According to the processing described using FIG. 4, as shown in the upper part of FIG. 5, if the endoscope 21 is connected thereto, when the video processor 22 receives an inspection end instruction signal from the server 3, the video processor 22 transmits an inspection end disable notification signal to the server 3 without switching the own processing state. In this way, it is possible to prevent the processing state of the video processor 22 from being switched from the inspection state to the non-inspection state in an unintended situation.

On the other hand, as shown in the lower part of FIG. 5, upon receiving an inspection end instruction signal from the server 3 when the endoscope 21 is not connected, the video processor 22 switches the own processing state from the inspection state to the non-inspection state and transmits an inspection end notification signal to the server 3. This allows the server 3 to appropriately switch the processing state of the video processor 22 from the inspection state to the non-inspection state and efficiently perform an inspection.

As described so far, according to the first embodiment, it is possible to appropriately switch the processing state of the video processor 22 from the inspection state to the non-inspection state remotely according to an inspection end instruction.

Note that in the first embodiment, for example, after the endoscope 21 is removed from the video processor 22, if the endoscope 21 to be used for the next inspection is connected to the video processor 22 before the processing state of the video processor 22 is switched from the inspection state to the non-inspection state, it is no longer possible to switch the processing state of the video processor 22 from the inspection state to the non-inspection state according to an inspection end instruction signal. To avoid this, the medical system 1 may be modified as follows.

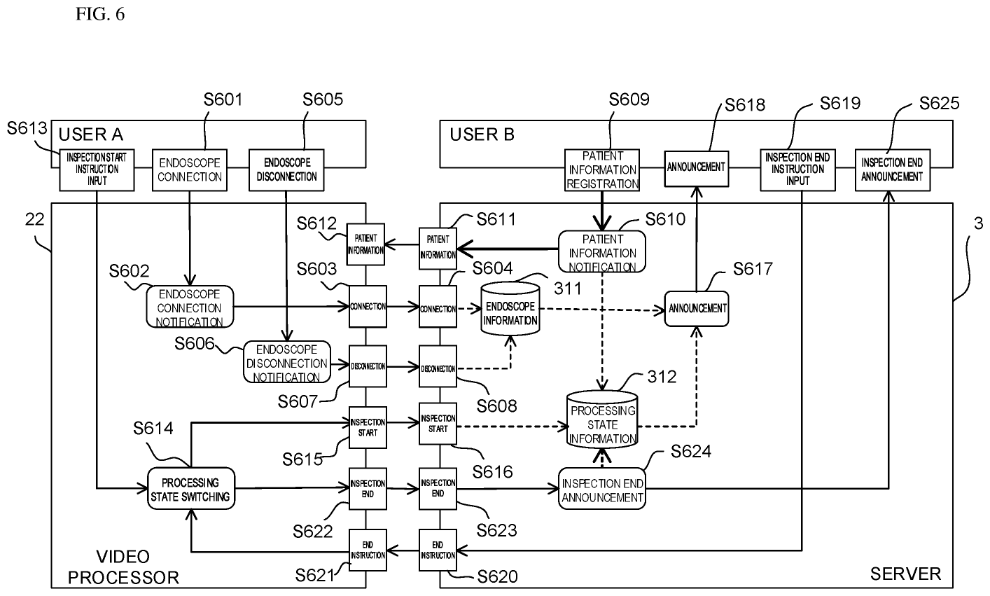

FIG. 6 is a diagram schematically illustrating an example of processing executed by the video processor and the server in a medical system according to a modification.

As shown in FIG. 6, in the present modification, if the user A in the inspection room connects the endoscope 21 to the video processor 22 (S601), the video processor 22 determines that the endoscope 21 is connected and transmits an endoscope connection notification signal for notifying the determination result to the server 3 (S602, S603). Upon receiving the endoscope connection notification signal from the video processor 22 (S604), the server 3 stores information indicating a state in which the endoscope 21 is connected in the endoscope information storage section 311 as information on the current connection state of the endoscope 21.

When the user A in the inspection room removes, that is, disconnects the endoscope 21 from the video processor (S605), the video processor 22 determines that the endoscope 21 is disconnected and transmits an endoscope disconnection notification signal for notifying the determination result to the server 3 (S606, S607). Upon receiving the endoscope disconnection notification signal from the video processor 22 (S608), the server 3 stores information indicating a state in which the endoscope 21 has been disconnected in the endoscope information storage section 311 as information on the current connection state of the endoscope 21.

When the user B outside the inspection room inputs patient information to perform a registration of patient information for the next inspection to the server 3 (S609), the server 3 transmits the patient information to the video processor 22 (S610, S611) and stores the patient information in the processing state information storage section 312. The patient information stored in the processing state information storage section 312 is stored in association with the information of the current processing state of the video processor 22 as will be described later. Upon receiving the patient information from the server 3 (S612), the video processor 22 registers the patient information as patient information of the patient for the next inspection. Note that the processing state of the video processor 22 at this time is assumed to be a non-inspection state.

When the user A in the inspection room inputs an inspection start instruction to the video processor 22 (S613), the video processor 22 switches the own processing state from the non-inspection state to the inspection state (S614) and transmits an inspection start notification signal for notifying an inspection start to the server 3 (S615). Note that the inspection start instruction is inputted to the video processor 22, for example, through operation on an operation panel provided for the video processor 22. Upon receiving the inspection start notification signal from the video processor 22 (S616), the server 3 stores information indicating an inspection state in the processing state information storage section 312 as information on the current processing state of the video processor 22. In this storage, information on the current processing state of the video processor 22 is associated with the patient information inputted and stored in S610.

The server 3 performs announcement based on information stored in the endoscope information storage section 311 and the processing state information storage section 312 (S617, S618). For example, the server 3 determines whether or not the endoscope 21 is disconnected from the video processor 22 based on the information on the current connection state of the endoscope 21 stored in the endoscope information storage section 311 and when it is determined that the endoscope 21 is disconnected, the server 3 announces the disconnection. The server 3 can also always announce the information on the current connection state of the endoscope 21 stored in the endoscope information storage section 311 and the current processing state of the video processor 22 and the patient information stored in association with each other in the processing state information storage section 312.

When the user B outside the inspection room who confirms through this announcement, that the endoscope 21 is disconnected from the video processor 22 inputs an inspection end instruction to the server 3 (S619), the server 3 transmits an inspection end instruction signal to the video processor 22 (S620). Upon receiving the inspection end instruction signal from the server 3 (S621), the video processor 22 switches the own processing state from the inspection state to the non-inspection state (S614) and transmits an inspection end notification signal for notifying an inspection end to the server 3 (S622). Upon receiving the inspection end notification signal from the video processor 22 (S623), the server 3 announces the inspection end, that is, that the endoscope system 2 is not under inspection to the user B (S624, S625). The server 3 stores information indicating the non-inspection state in the processing state information storage section 312 as information on the current processing state of the video processor 22.

The user B who confirms that the endoscope system 2 is not under inspection through this announcement inputs patient information to perform a registration of patient information for the next inspection (S609), and processing similar to the aforementioned processing is performed hereinafter.

According to the present modification, the server 3 can manage the current connection state of the endoscope 21 and the current processing state of the video processor 22. Since the user B outside the inspection room can confirm through the announcement from the server 3 that the endoscope 21 is disconnected from the video processor 22, the user B can execute an inspection end instruction and perform a registration of patient information at appropriate timing.

Note that in the present modification, when the video processor 22 receives an inspection end instruction signal from the server 3 (S621), the video processor 22 may determine whether or not the endoscope 21 is connected to the connection section 221. The video processor 22 may switch the own processing state from the inspection state to the non-inspection state only when it is determined that the endoscope 21 is not connected to the connection section 221 (S614).

### Second Embodiment

Some conventional endoscope systems set inspection information distributed from a network apparatus such as a server in a video processor as inspection information for the next inspection, cause the set inspection information to be displayed on an observation monitor so that the user can confirm the inspection information for the next inspection. However, the observation monitor needs to display the endoscopic image on a main screen, but since space of the display region for character information is limited, the character information to be always displayed on the observation monitor is limited to patient information (patient ID (identifier) and patient name) included in the inspection information. For this reason, it is not possible to confirm information other than the patient information such as a scheduled patient inspection date and a type of inspection included in the inspection information set in the video processor in the inspection room, the user may not notice errors in the inspection information distributed from the network apparatus, for example, that the scheduled inspection date is different, and the user may mistake one inspection for another.

It is for this reason that there is a demand for a mechanism that allows the user to confirm inspection information such as the scheduled inspection date and the inspection type without affecting the display region of character information on the observation monitor.

The second embodiment proposes the mechanism.

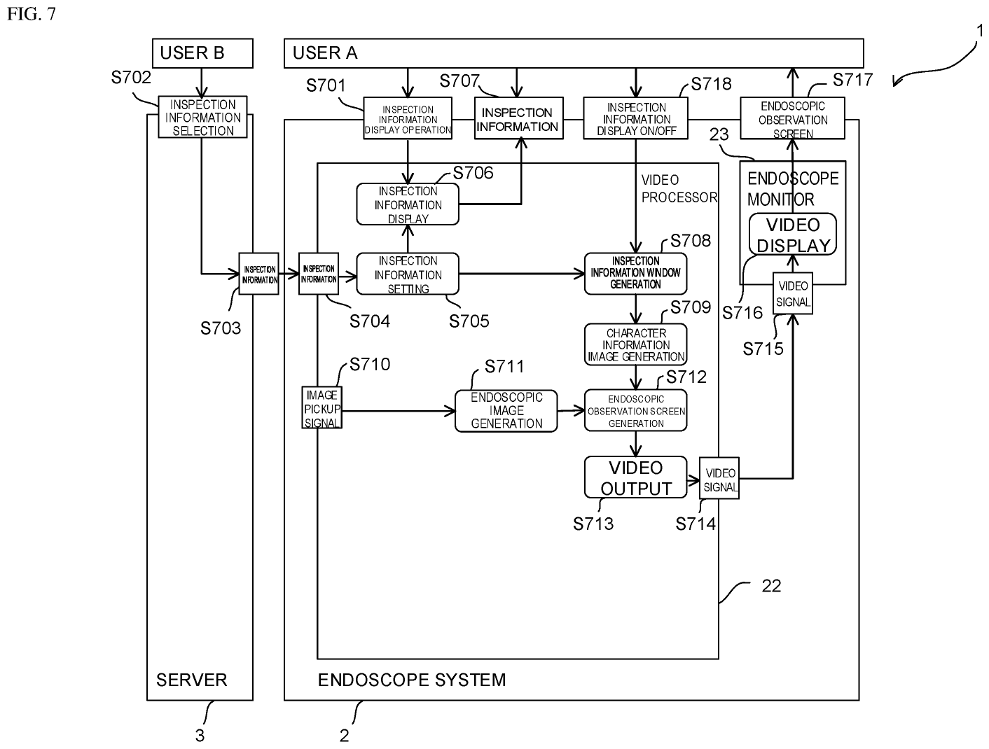

FIG. 7 is a diagram schematically illustrating an example of processing executed by a medical system according to a second embodiment. Note that the configuration of the medical system according to the second embodiment is basically the same as that of the first embodiment, and a medical system, a server, an endoscope system and a video processor according to the second embodiment are assigned the same reference numerals as those in the first embodiment.

As shown in FIG. 7, in this processing, the user A in the inspection room operates the operation panel of the video processor 22 (S701) to set a display destination of inspection information in the video processor 22 first. Examples of the display destination that can be set in the present embodiment include the display panel of the video processor 22 or an endoscope monitor 23 connected to the video processor 22.

After that, when the user B outside the inspection room performs an input to select inspection information for the next inspection to the server 3 (S702), the server 3 transmits inspection information selected according to the input to the video processor 22 (S703).

Upon receiving the inspection information from the server 3 (S704), the video processor 22 sets the received inspection information as inspection information for the next inspection (S705).

Next, if the set display destination of the inspection information is the own display panel, the video processor 22 displays the inspection information set in S705 on the own display panel (S706, S707). On the other hand, if the set display destination of the inspection information is the endoscope monitor 23, the video processor 22 generates an inspection information window including the inspection information set in S705 (S708). Note that the inspection information displayed on the display panel of the video processor 22 in S706 and S707, and the inspection information included in the inspection information window generated in S708 may be all the inspection information set in S705 or partial information including at least scheduled inspection dates or inspection types or the like or inspection information other than the patient information.

Next, the video processor 22 generates a character information image based on character information relating to characters to be displayed in a character information display region of the endoscope monitor 23 (S709). The character information includes the patient information included in the inspection information set in S705.

The video processor 22 acquires an image pickup signal from the endoscope 21 (S710) and generates an endoscopic image (S711). The endoscopic image generated here is an image to be displayed in the endoscopic image display region of the endoscope monitor 23.

The video processor 22 generates an endoscopic observation screen as shown below according to the display destination of the set inspection information (S712).

When the set inspection information display destination is the own display panel, the video processor 22 generates an endoscopic observation screen based on the character information image generated in S709 and the endoscopic image generated in S711. This endoscopic observation screen corresponds to a conventional endoscopic observation screen.

On the other hand, when the set inspection information display destination is the endoscope monitor 23, the video processor 22 generates an endoscopic observation screen based on the inspection information window generated in S708, the character information image generated in S709 and the endoscopic image generated in S711. This endoscopic observation screen is a screen corresponding to a conventional endoscopic observation screen on which an inspection information window is superimposed.

Next, the video processor 22 outputs the generated endoscopic observation screen to the endoscope monitor 23 as a video signal (S713, S714).

Upon receiving the video signal from the video processor 22 (S715), the endoscope monitor 23 displays an endoscopic observation screen corresponding to the video signal (S716, S717). When the inspection information display destination set by the user A is the display panel of the video processor 22, the endoscope monitor 23 displays the character information including the patient information and the endoscopic image. On the other hand, when the inspection information display destination set by the user A is the endoscope monitor 23, the endoscope monitor 23 displays character information including the patient information and the endoscopic image, and also displays inspection information in a window.

Note that when the endoscope monitor 23 is set as the inspection information display destination, the user A in the inspection room can turn ON/OFF the window display of the inspection information on the endoscope monitor 23 by operating the operation panel of the video processor 22 (S718). For example, when the user A in the inspection room performs operation of turning OFF the window display, the video processor 22 stops the generation of the inspection information window in S708 until operation of turning ON the window display is performed after that, and in S712, the video processor 22 generates an endoscopic observation screen based on the character information image generated in S709 and the endoscopic image generated in S711.

As described so far, according to the second embodiment, it is possible to display the inspection information on the display panel of the video processor 22 according to the user's setting. In this case, the user can confirm inspection information such as the scheduled inspection date and the inspection type without affecting the display region of character information on the endoscope monitor 23.

Furthermore, it is also possible to display the inspection information on the endoscope monitor 23 in a window according to the user's setting. In this case, although the inspection information is displayed in the window on the endoscopic image and character information displayed on the endoscope monitor 23, the window display can be turned ON/OFF by the user's operation. Therefore, the user can confirm inspection information such as the scheduled inspection date and the inspection type without affecting the confirmation of the endoscopic image and character information displayed on the endoscope monitor 23.

As described so far, the present embodiment is not limited to the above-described embodiments as they are, but the components thereof may be modified and embodied without departing from the gist of the present invention in an implementation stage. Furthermore, various embodiments may be formed by combining the plurality of components disclosed in the above-described embodiments as appropriate. For example, some of all the components disclosed in the embodiments may be deleted. Furthermore, components across different embodiments may be combined as appropriate.

What is claimed is:

1. An endoscope apparatus comprising:
a connection interface to which an endoscope is connected;
a communication interface that transmits and receives signals to and from an external apparatus; and
a computation device includes a processor configured to:
determine whether the endoscope is connected to the connection interface;
switch a processing state of the endoscope apparatus between an inspection state to a non-inspection state; and
control switching the processing state from the inspection state to the non-inspection state upon acquiring an instruction signal from the external apparatus when the processor determines that the endoscope is not connected to the connection interface.

2. The endoscope apparatus according to claim 1, wherein
the computation device does not perform control to switch the processing state from the inspection state to the non-inspection state when the instruction signal is acquired and it is determined that the endoscope is connected to the connection interface.

3. The endoscope apparatus according to claim 1, wherein
the communication interface transmits a notification signal for notifying the external apparatus that the endoscope apparatus is under inspection when the computation device acquires the instruction signal and it is determined that the endoscope is connected to the connection interface.

4. The endoscope apparatus according to claim 1, wherein
the endoscope apparatus is disposed in an inspection room, and
the external apparatus is disposed outside the inspection room.

5. The endoscope apparatus according to claim 1, wherein
the inspection state represents a state that the endoscope apparatus performs processing of generating data and recording the data and patient information relating to a patient under inspection in association with each other.

6. A processing state switching method executed by a processor provided in an endoscope apparatus, the processor configured to execute:
determining whether an endoscope is connected to the endoscope apparatus based on an instruction signal providing an instruction for switching of a processing state of the endoscope apparatus from an inspection state to a non-inspection state from an external apparatus; and
switching the processing state of the endoscope apparatus from an inspection state to a non-inspection state when the endoscope is not connected to the endoscope apparatus.

7. A medical system comprising an endoscope apparatus and a network apparatus,
the endoscope apparatus comprising:
a connection interface to which an endoscope is connected;
a first computation device including a processor configured to:
determine whether the endoscope is connected to the connection interface; and
switch a processing state of the endoscope apparatus between an inspection state and a non-inspection state,
a first communication interface configured to transmit and receive signals to and from the network apparatus, wherein
the network apparatus comprises:
a second communication interface configured to transmit and receive signals to and from the endoscope apparatus;
a second computation device that includes a processor configured whether the endoscope is disconnected from the endoscope apparatus;
an output device configured to inform a user that the endoscope is disconnected from the endoscope apparatus when the second computation device determines that the endoscope is disconnected from the endoscope apparatus; and
an input device that inputs an instruction,
the first communication interface being configured to transmit a notification signal for notifying a determination result of the first computation device to the network apparatus,
the processor of the second computation device being configured to make the determination based on the notification signal acquired from the endoscope apparatus via the second communication interface,
the second communication interface configured to transmit an instruction signal for instructing switching of the processing state from the inspection state to the non-inspection state to the endoscope apparatus when an inspection end instruction is inputted via the input device, and
the processor of the first computation device being configured to switch the processing state from the inspection state to the non-inspection state when the instruction signal is acquired from the network apparatus via the first communication interface.

8. An endoscope apparatus comprising:
a connection interface to which an endoscope is connected;
a computation device including a processor configured to:
determine whether or not the endoscope is connected to the connection interface, and
switch a processing state of the endoscope apparatus from an inspection state to a non-inspection state or from a non-inspection state to an inspection state, and
a communication interface configured to transmit and receive signals to and from an external apparatus, wherein
the communication interface transmits a notification signal configured to notify a determination result of the computation device to the external apparatus, and
the computation device configured to switch the processing state from the inspection state to the non-inspection state when an instruction signal is received from the external apparatus via the communication interface.

9. A network apparatus comprising:
a communication interface configured to transmit and receive signals to and from an endoscope apparatus;
a computation device that includes a processor configured to determine whether or not an endoscope is disconnected from the endoscope apparatus;
an output device configured to inform a user that the endoscope is disconnected from the endoscope apparatus when the computation device determines that the endoscope is disconnected from the endoscope apparatus; and
an input device configured to input an instruction, wherein
the computation device makes the determination based on a notification signal configured to notify a determination result as to whether or not the endoscope is connected to the connection interface of the endoscope apparatus acquired from the endoscope apparatus via the communication interface, and
the communication interface configured to transmit an instruction signal configured to instruct the computation device to switch a processing state of the endoscope apparatus from an inspection state to a non-inspection state to the endoscope apparatus when an inspection end instruction is inputted via the input device.
